(19) United States
(12) Patent Application Publication
(10) Pub. No.: US 2007/0212787 A1
(43) Pub. Date: Sep. 13, 2007

(54) METHOD OF QUANTITATIVELY MEASURING TRACE AMOUNTS OF METALS IN POLYMER SAMPLES

(75) Inventors: Michael Riess, Taunusstein (DE); Elena Schick, Mainz-Kastel (DE)

Correspondence Address: MOTOROLA, INC., 1303 EAST ALGONQUIN ROAD, IL01/3RD, SCHAUMBURG, IL, 60196, US

(73) Assignee: MOTOROLA, INC., Schaumburg, IL (US)

(21) Appl. No.: 11/276,672

(22) Filed: Mar. 9, 2006

(51) Int. Cl. G01N 33/20

(52) U.S. Cl. 436/73

(57) ABSTRACT

A method of quantitatively measuring trace amounts of metals in polymer samples using x-rays to fluoresce and detect metals that are present in the polymer sample is disclosed. The polymer sample is ground to a powder and formed into a thin film by heat and pressure. The film is then analyzed using energy dispersive x-ray fluorescence spectrometry, and the data representing the amounts of the various metals present in the sample is multiplied by one or more correlation factors that have been determined from measurement of characterized polymers that have similar composition to the polymer sample and that bracket a range of metal concentrations in the sample.

### FIELD OF THE INVENTION

This invention relates generally to quantitative chemical analysis. More particularly, this invention relates to quantitative analysis of polymers by energy dispersive x-ray fluorescence.

### BACKGROUND

Legislation in the European Union (EU) has been enacted to reduce the level of hazardous chemicals in the environment. The Restriction of certain Hazardous Substances (ROHS) act has targeted materials such as lead, cadmium, mercury, chromium VI and some brominated flame retardants used in electronic devices. In order to comply with these enacted regulations, a method to detect and identify these and other hazardous chemicals in electronic devices is needed. Such a method should be fast and accurate to enable rapid testing and short turnaround times in keeping with the ‘time to market’ requirements of the global electronics industry. The detection method also needs to be highly cost effective and it should not have any adverse impact on the environment by using other hazardous substances in the detection procedure.

Prior art methods for analysis of trace amounts of metals in polymers have focused on methodologies such as wet digestion (microwave, sulfuric acid digestion, etc.) or by the well-known internal standard or external standard techniques using commercial standards. Although these methods are time-tested and internationally accepted, they are time consuming and costly, and require sophisticated sample preparation and measurement.

### BRIEF DESCRIPTION OF THE DRAWINGS

The accompanying FIGURE, which together with the detailed description below are incorporated in and form part of the specification, serves to further illustrate various embodiments and to explain various principles and advantages all in accordance with the present invention.

FIG. 1 is a flow chart in accordance with certain embodiments of the present invention.

### DETAILED DESCRIPTION

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting; but rather, to provide an understandable description of the invention. The terms a or an, as used herein, are defined as one or more than one. The term plurality, as used herein, is defined as two or more than two. The term another, as used herein, is defined as at least a second or more. The terms including and/or having, as used herein, are defined as comprising (i.e., open language).

A method of quantitatively measuring trace amounts of metals in polymer samples uses energy dispersive x-ray fluorescence (EDXRF) spectrometry to fluoresce and detect metals that are present in the polymer sample. The polymer sample is ground to a powder and formed into a thin film by heat and pressure. The film is then analyzed using EDXRF, and the data representing the amounts of the various metals present in the sample is multiplied by one or more correlation factors that have been determined from measurement of characterized polymers that have similar composition to the polymer sample and that bracket a range of metal concentrations in the sample.

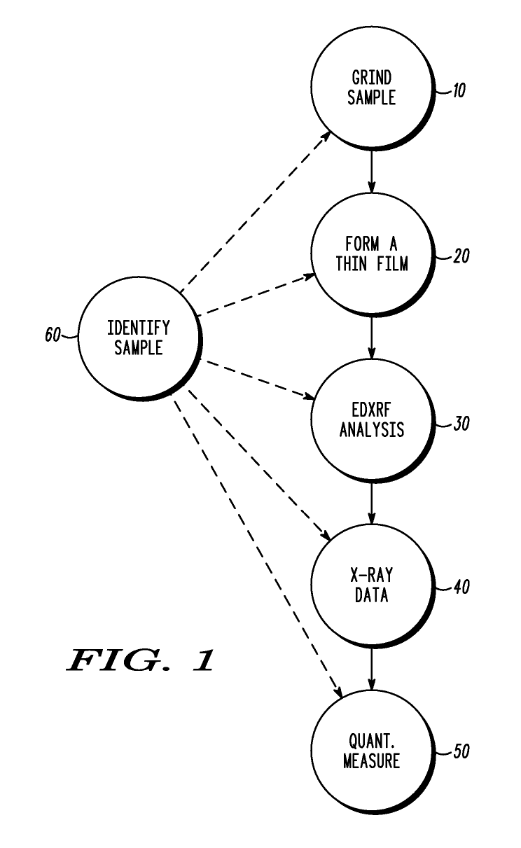

Referring now to FIG. 1, a flow chart depicting the a methodology of one embodiment of the invention, a sample of a polymer that contains trace amounts of metals of interest is obtained and ground to a fine powder 10 in a laboratory grinder, until a particle size of about 1 millimeter or less is reached. This is the typical size, although one skilled in the art will find that other size ranges may be used. The ground polymer sample is then formed 20 into a thin film under heat and/or pressure in a laboratory press. Typically, when thermoplastics are used, the powder is heated to a temperature above the melting point of the polymer, or at least above the glass transition point of the polymer, so as to allow the powder to flow into a continuous film. The sample is also exposed to pressure to aid in the creation of a uniform and thin film that is between 10 and 1000 micrometers thick, and we find that films of about 250 micrometers thickness serve well. A distance ring or other fixture may be used to define the thickness of the film. Once the powder has been formed into a film, the pressed polymer film is analyzed using energy dispersive x-ray fluorescence spectrometry 30 to detect the metals of interest that may be present in the polymer sample. The x-ray fluorescence spectrometer consists of a primary x-ray tube that emits x-rays of known energy. This primary x-ray beam is directed onto the surface of the thin film, and any elements in the sample having excitation energy below that of the primary beam energy will be fluoresced by the primary beam. The fluoresced x-rays are then detected and counted by an x-ray detector, such as an energy dispersive spectrometer. The energy dispersive detector allows for efficient and simultaneous detection of elements having a wide range of x-ray energies, and is most useful for screening a set of elements. Spectral acquisition times can be as short as a few seconds to greater than 1000 seconds depending on the concentration of the elements being measured. The EDXRF provides information and data that is representative of the amount of x-rays detected for each fluoresced element. Although most commercial EDXRF systems have software that attempts to calculate the weight percentage of the various detected elements, matrix effects in the sample cause these calculations to be in significant error, and thus, even though the EDXRF output might display percentages of the various elements present, it is not accurate enough to be relied upon.

A more accurate determination of the actual amount of the various metals in the polymer sample present in weight percent is obtained by multiplying the data 40 that is representative of the amount of x-rays collected for each element by one or more correlation or calibration factors 50. These factors are determined by measurement of known, characterized polymers that have similar composition to the polymer sample and that bracket a range of metal concentrations in the polymer sample. This, of course, requires that one knows the identity or type of polymer(s) present in the polymer sample. The identity of the polymer sample can be obtained in a number of ways, and although it can be identified at any point in the procedure prior to measuring 50, we find it most convenient to identify the polymer at the beginning of the analysis, that is, prior to grinding to a powder 10. The identity of the polymer can be determined 60 by infrared spectroscopy analysis, other well-known laboratory analysis methodologies, it may already be known by the analyst, or the identity can be provided by others (such as the manufacturer of the polymer). The plurality of calibration factors obtained on the known polymer samples can be combined into one or more calibration curves that plot, for example, weight percentage of a detected metal as a function of the information provided by the EDXRF spectrometer.

In summary, without intending to limit the scope of the invention, trace amounts of metals present in polymers can be accurately determined according to a method consistent with certain embodiments of the invention by forming a thin film of the sample under heat and pressure, analyzing the film using EDXRF, and multiplying the data representing the amounts of the various metals present in the sample by one or more correlation factors that have been determined from measurement of characterized polymers that have similar composition to the polymer sample. While the invention has been described in conjunction with specific embodiments, it is evident that many alternatives, modifications, permutations and variations will become apparent to those of ordinary skill in the art in light of the foregoing description. Accordingly, it is intended that the present invention embrace all such alternatives, modifications and variations as fall within the scope of the appended claims.

What is claimed is:

1. A method of quantitatively measuring trace amounts of metals in polymer samples of a polymer, comprising:
providing the identity of a polymer sample;
providing a thin film of the polymer sample;
analyzing the thin film by means of energy dispersive x-ray fluorescence spectrometry so as to fluoresce and detect metals that are present in the polymer sample; and
quantitatively measuring the amount(s) of detected metals in the thin film of the identified polymer sample by means of a calibration curve that is uniquely determined for the polymer.

2. The method as described in claim 1, wherein providing a thin film comprises grinding the polymer sample to a powder and forming the thin film by heating and pressing the powder.

3. The method as described in claim 1, wherein providing the identity comprises identifying the polymer sample by means of infrared spectroscopy.

4. The method as described in claim 1, wherein providing the identity is performed by others.

5. The method as described in claim 2, wherein providing a thin film further comprises providing a thin film that is less than 500 micrometers thick.

6. The method as described in claim 1, wherein the calibration curve is assembled from measurement of polymers that have similar composition to the polymer sample.

7. The method as described in claim 1, wherein quantitatively measuring comprises multiplying data provided by the energy dispersive x-ray fluorescence spectrometer by a calibration factor determined from measurement of characterized polymers that have similar composition to the polymer sample and that bracket a range of metal concentrations in the polymer sample.

8. A method of quantitatively measuring trace amounts of metals in polymer samples, comprising:
providing the identity of a polymer sample;
grinding the polymer sample to a powder and forming a thin film by heating and pressing the powder;
analyzing the thin film by means of energy dispersive x-ray fluorescence spectrometry so as to fluoresce and detect metals that are present in the polymer sample; and
quantitatively measuring the amount(s) of detected metals in the thin film of the polymer sample by multiplying data provided by the energy dispersive x-ray fluorescence spectrometer by a calibration factor determined from measurement of characterized polymers that have similar composition to the polymer sample and that bracket a range of metal concentrations in the polymer sample.

9. The method as described in claim 8, wherein providing the identity comprises identifying the polymer sample by means of infrared spectroscopy.

10. The method as described in claim 8, wherein providing the identity is performed by others.

11. A method of quantitatively measuring trace amounts of metals in a polymer sample, comprising:
grinding the polymer sample to a powder and forming a thin film under heat and pressure to melt the powder;
analyzing the thin film by means of energy dispersive x-ray fluorescence spectrometry wherein x-rays are fluoresced from metals that are present in the thin film and detected and counted by an x-ray detector to produce a first set of data representative of the quantitative amount of metals that are present in the thin film; and
calculating the amount(s) of detected metals in the thin film by multiplying the first set of data by factors that are derived from energy dispersive x-ray fluorescence measurement of polymers that have composition similar to the polymer sample.

12. The method as described in claim 11, wherein the thin film formed under heat and pressure is less than 500 micrometers thick.

13. The method as described in claim 11, wherein the polymer sample comprises a thermoplastic polymer.

14. The method as described in claim 11, wherein calculating comprises measuring polymers that have composition similar to the polymer sample and that bracket a range of metal concentrations in the polymer sample.
